(19) United States
(12) Patent Application Publication
(10) Pub. No.: US 2024/0010993 A1
(43) Pub. Date: Jan. 11, 2024

(54) CELL CULTURE CHAMBER AND METHOD FOR CULTURING CELLS AND FOR THE IN VITRO PRODUCTION OF CELL LAYERS AND ORGAN MODELS

(71) Applicant: Dynamic42 GmbH, Jena (DE)

(72) Inventors: Knut Rennert, Jena (DE); Martin Raasch, Weimar (DE); Nancy Blaurock-Moeller, Ilmenau (DE); Katja Graf, Jena (DE); Nader Abdo, Jena (DE)

(21) Appl. No.: 18/469,397

(22) Filed: Sep. 18, 2023

Related U.S. Application Data
(63) Continuation of application No. PCT/EP2022/057258, filed on Mar. 18, 2022.

(30) Foreign Application Priority Data
Mar. 19, 2021 (DE) 10 2021 106 915.7

(51) Int. Cl. C12N 5/071; C12M 3/00; C12M 1/12; C12M 1/00; C12M 1/42

(52) U.S. Cl. CPC C12N 5/0697; C12M 21/08; C12M 25/04; C12M 29/06; C12M 35/08; C12N 2533/52; C12N 2533/54; C12N 2513/00; C12N 2502/00

(57) ABSTRACT

A cell culture chamber is for the in vitro production and cultivation of cell layers and organ models with two first and second channels arranged one above the other and separated from one another by a porous membrane with two side surfaces and through which flow can pass, wherein a cell substrate is formed in each case by the side surfaces of the membrane. The cell culture chamber is characterized in that at least the inner walls of the first and the second channels consist of polybutylene terephthalate. Further, a method is for cultivating human or animal cells, and in particular liver sinusoidal endothelial cells, alone and in co-culture with hepatocytes and immune cells.

### CROSS-REFERENCE TO RELATED APPLICATIONS

This application is a continuation application of international patent application PCT/EP2022/057258, filed Mar. 18, 2022, designating the United States and claiming priority from German application 10 2021 106 915.7, filed Mar. 19, 2021, and the entire content of both applications is incorporated herein by reference.

### TECHNICAL FIELD

The disclosure relates to a cell culture chamber for cultivating cells and for the in vitro production of cell layers as well as organ models, and a method for culturing and for improved functional integrity, in particular, of liver sinusoidal endothelial cells (LSEC) alone and also in co-culture with liver-specific cell types.

### BACKGROUND

Devices, and in particular cell culture chambers, are known from the prior art that allow an in vitro culturing and investigation of cell cultures under perfusing conditions. In this, the particular cell cultures can be flushed or incubated with liquids or also gas and aerosol mixtures, and conditions can be accordingly simulated which very closely correspond to in vivo physiological conditions. In addition, cell culture chambers of this kind are suitable for investigating effects and any occurring interactions between one or more cell cultures and a medium or several media and test substances contained in the medium (for example, in the areas of pharmacokinetics (PK) and pharmacodynamics (PD), studies of absorption, distribution, metabolism, excretion and toxicology (ADMET), substance sensitivity tests, and substance safety tests).

Cell culture chambers are described, for example, in US 2017/0226457. These include two channels which are separated from one another by a porous membrane. As the material for such cell culture chambers, plastics are often used, which are biocompatible and can also easily be produced in complex forms. In particular, such materials are suitable for being processed via various injection-molding techniques. Examples of such plastics are polyurethanes (PU), polyimides, styrenes (SEBS), polypropylenes (PP), polystyrenes (PS), polycarbonates (PC), and cyclic polyolefins (COP and COC). In addition, cell culture chambers are produced from polydimethylsiloxanes (PDMS) and used, for example, for culturing complex cell/organ models (for example, WO 2017/096282 A1, WO 2019/164962 A1). However, the use of PDMS-based chips for complex cell/organ models is complicated, since the PDMS, in contrast to biocompatible plastics that can be injection-molded, has to be activated beforehand to be suitable for cell cultures at all. This is accomplished, for example, by introducing activation solutions in conjunction with UV irradiation and several washing steps. These preliminary procedures are not easy to perform in every laboratory by the end user and harbor a certain potential for errors before the actual cell culture starts.

In order to influence as little as possible the systems of biological structures and media to be investigated, the materials of the cell culture chambers should ideally be inert. However, for example, it is known from the frequently used material PDMS that this has high binding capacities to some chemical compounds (Auner, et al., (2019): Chemical-PDMS-binding kinetics and implications for bioavailability in microfluidic devices; Lab Chip 19: 864-874). This bond-forming capacity can have a difficult-to-estimate influence on experiments that are carried out with cell culture chambers made of PDMS. For example, substances with a log P greater than 1.8 and a hydrogen donor count of 0 are adsorbed strongly on PDMS, which makes active substance testing and interpretation of the obtained data considerably more difficult. For example, the substance propiconazole has a log P of 3.72 and a hydrogen donor count of 0. It is very hydrophobic and can only be detected at less than 30% of the starting concentration in the culture medium after 24 hours in PDMS chips, since it binds irreversibly to PDMS (Auner et al., 2019).

### SUMMARY

It is an object of the disclosure to provide an improved cell culture chamber and its use, via which the disadvantages known from the prior art are reduced.

The object is, for example, achieved via a cell culture chamber including:
a first channel including an inner wall, through which a first flow can pass; a second channel including an inner wall, through which a second flow can pass; and a porous membrane; wherein the first and second channels are arranged one above the other and are separated from one another by the membrane, wherein the membrane has a first side surface oriented towards the first channel and a second side surface oriented towards the second channel, wherein the first and second side surfaces form a cell substrate, and wherein the inner walls of the first channel and the second channel consist of polybutylene terephthalate (PBT).

The object is achieved with a cell culture chamber for cultivating cells and the in vitro production of cell layers and organ models. The cell culture chamber according to the disclosure has at least two channels which are arranged one above the other and can be separated from one another by a porous membrane with two side surfaces through which a flow can pass, wherein a cell substrate is formed in each case by the side surfaces of the membrane.

The device is characterized in that at least the inner walls of the first and second channels are made of polybutylene terephthalate (PBT).

PBT is a polymer which is used to produce products that are subject to a high mechanical load and/or which repeatedly come into contact with hot media. Typical uses of PBT are, for example, plain bearings, valve parts, screws, parts for household appliances such as coffee machines, egg cookers, toasters, or hair dryers. PBT is very suitable for injection-molding processing.

This material, which is unusual for use in cell culture chambers, showed very low bond-forming capacity in tests, compared to a series of components of the media used, such that an influence of the material of the cell culture chamber, and in particular of the (preliminary) culture chambers, on the tests taking place in such a cell culture chamber can be advantageously reduced. For example, in tests for active substance adsorption—inter alia, with propiconazole and troglitazone—the inventors have found that PBT is very suitable for active ingredient tests of substances up to the log P of 3.72 (log P of propiconazole: 3.72; log P of troglitazone: 3.60). After an incubation period of 24 h, at least 80% of the starting concentration of the propiconazole or the troglitazone is detectable in the culture medium (cf. FIG. 3A, FIG. 3B).

The cell culture chamber according to the disclosure can be used for the cultivation of cell cultures and organ models as well as for testing them (for example, real-time observation using optical solutions such as microscopy). In this case, the cell cultures/organ models with media of a defined composition can be supplied under also known and possibly regulatable ambient conditions (for example, temperature, air pressure, flow rate and shear, relative orientation of the cell culture chamber, oxygen, pH). Advantageously, the cell culture chamber can also be used for cultivating at least two different cell types. The cultivation of at least two different cell types is also referred to herein as a co-culture or co-culturing. Such a co-culture can let the model environment further approach in vivo conditions. Such a co-culture of at least two different cell types in the cell culture chamber establishes an organ model in the sense of the disclosure.

A specific use of the cell culture chamber according to the disclosure is to culture so-called liver sinusoidal endothelial cells (LSEC) and investigate them as needed. Advantageously, the LSEC in the cell culture chamber can also be cultured in co-culture with other liver cells such as, in particular, hepatocytes, Kupffer cells/macrophages, and/or stellate cells. LSEC, alone and in co-culture with other liver cells, are an important test system for the investigation of a variety of functions and interactions of the liver as one of the central metabolic organs of the body—in particular, of mammals. However, the cultivation of these LSEC alone as well as in co-culture with other liver cells is very demanding. A brief overview will be given below.

Liver sinusoidal endothelial cells are specialized endothelial cells of the liver which are characterized by the lack of a pronounced basal membrane and the presence of small open pores called fenestrae. The unique permeable structure of the LSEC enables the blood plasma to freely access the Disse space located between the LSEC and the hepatocytes. As a result, hepatocytes can exchange small molecules, for example, nutrients, without direct contact with the blood stream. In addition, LSEC in the body perform specific functions such as the so-called clearance of macromolecules, the production of coagulation factors, and the adherence of immune cells, for example, when there is liver damage. Furthermore, these are characterized by specific markers such as factor VIII, stabilin-2, LSECtin, and CD32b (see Table 1). It is believed that LSEC are directly involved in drug-induced liver toxicity, which is why they are interesting for more in-depth drug testing.

TABLE 1
Known liver sinusoidal endothelial cell markers and their function

| Markers | Designation | Function | Detection Method |
|---|---|---|---|
| CD31 | Platelet | Control of | Immunofluorescence |
| (PECAM-1) | endothelial | leukocyte | (IF), flow cytometry, |
| | cell adhesion | transmigration | fluorescence activated |
| | molecule | | cell sorting (FACS) |
| CD32b | FcgRIIb | Inhibitor | Immunohistochemistry |
| | | receptor | (IHC), IF Western |
| | | for IgG | blotting (WB), FACS |
| CD206 | Mannose | Phagocytosis of | IF, WB |
| | receptor | pathogens | |
| Factor | Blood clotting | Support for blood | IF |
| VIII | factor VIII | clotting, co-factor | |
| | | for factor IXa | |
| FcRn | Neonatal IgG | Recycling of IgG | IF |
| | Fc receptor | and serum albumin | |
| LSECtin | Sinusoidal | Lectin receptor, | IF |
| (CLEC4G) | endothelial | pathogen-binding | |
| | cell C-type | factor | |
| | lectin | | |
| L-SIGN | Liver/lymph | Type II-integral | IHC, IF |
| (CD209L or | node-specific, | membrane protein, | FACS |
| CLEC4M) | intracellular | pathogen | |
| | adhesion | recognition | |
| | molecule 3- | receptor | |
| | binding non- | | |
| | integrin | | |
| LYVE-1 | Endothelial | Not understood, | IHC, IF |
| | hyaluronic | possible function: | FACS |
| | acid | hyaluronic acid | |
| | receptor-1 | transport and | |
| | | turnover, | |
| | | promotion of HA | |
| | | localization on | |
| | | endothelial surface | |
| MHC I | HLA-A, | Binds and presents | IF, FACS |
| | -B, -C | peptides of | |
| | | proteasome | |
| | | (intracellularly) | |
| | | degraded proteins, | |
| | | presents peptides for | |
| | | cytotoxic T-cells | |
| MHC II | HLA-DR- | Presentation of | IF, FACS |
| | DP-DQ | lysosomal-degraded | |
| | | extracellular | |
| | | pathogens for | |
| | | T-helper cells | |
| Stabilin-2 | | Angiogenesis, | IHC, IF |
| | | scavenger receptor, | WB |
| | | lymphocyte homing, | |
| | | cell adhesion | |
| VAP-1 | Vascular | Lymphocyte adhesion | IF |
| | adhesion | molecule, monoamine | |
| | protein-1; | oxidase | |
| | AOC3 | | |
| vWF | von | Carrier protein of | IF |
| | Willebrand | blood coagulation | |
| | factor | factor VIII | |
| Fenestrae | Pore with | Clearance of | Scanning |
| | diameters | old blood | electron |
| | between | components | microscopy |
| | 100-150 nm, | | (SEM) |
| | cover 20% | | |
| | of LSEC | | |
| | surface | | |

Special requirements when using LSEC as a model system are due to the fact that the LSEC generally loses its functionality and specific markers (see Table 1) after isolation within a few hours to days. They are then, for example, no longer or only slightly suitable for checking the toxicity of active substances—for example, when checking the contribution of the immune cells to hepatotoxicity. The disclosure proposes solutions via which the functionalities of LSEC alone and in co-culture with other liver-specific cell types can be retained much longer. For this purpose, for example, the channels can be configured such that vascular conditions of a blood vessel can be simulated in the first channel with the aid of LSEC. The LSEC can be cultured alone or in co-culture with other cells—in particular, Kupffer cells.

Kupffer cells, the resident macrophages of the liver, are located in vivo in the hepatic sinusoid, thereby performing a monitoring function by enabling the liver to respond to pathogens and damage. Kupffer cells are able to precisely regulate, for example, drug-induced inflammation or phenotypic changes, which results in a differential expression and secretion of pro- or anti-inflammatory mediators (for example, IL-1?, TNF, IL-6, IL-10). This critical regulation of the inflammation reaction makes them an important component of in vitro models of liver diseases, and of in vitro screening models for drug toxicity that results from inflammation-related downregulation of drug-metabolizing enzymes and/or drug transporters. Kupffer cells/macrophages thereby play an important role in the development of side effects of substances/active ingredients. In the liver, Kupffer cells/macrophages together with LSEC regulate the immune response. They play an important role in the development of non-alcoholic fatty liver disease (NAFLD) and non-alcoholic steatohepatitis (NASH), for example. There are also indications that Kupffer cells protect the liver against drug-induced liver injury (DILI) by some drugs in that they release hepatoprotective cytokines, including IL-10 and IL-6. With regard to other drugs, Kupffer cells have been shown to significantly contribute to DILI due to the release of pro-inflammatory cytokines, including TNF, IL-1?, and IL-6, which may be either protective or damaging depending upon the context. In some instances, the inclination of a drug to induce DILI, can only be detected in vitro when Kupffer cells are present and directed towards a pro-inflammatory reaction. The cell culture chamber according to the disclosure offers the great advantage that Kupffer cells together with LSEC can be co-cultured under physiological conditions. Targeted in vitro DILI tests under conditions approximating the body are accordingly made possible.

Hepatic conditions can be simulated in the second channel. The hepatic conditions can be produced, for example, with the aid of hepatocytes. LSEC and hepatocytes in co-culture can form an organ model of a mammalian liver. The hepatocytes can be cultured alone or in co-culture with other cells—in particular, stellate cells.

Stellate cells (HSC's) in a “resting” state are described as stores of Vitamin A and as antigen-presenting cells in the liver. An activation of the stellate cells is the critical step in developing liver fibrosis. It is associated with the loss of Vitamin A storage and results in transdifferentiation under the stimulation of cellular mediators, cytokines, and chemokines from injured or inflammatory cells. In the initial stage of fibrogenesis, HSC's transform into proliferative and contractile myofibroblasts. These accelerate the secretion of extracellular matrix and reduce the degradation of the extracellular matrix elements, which ultimately leads to fibrogenesis. Together with the Kupffer cells, they make a significant contribution to the development of NAFLD, NASH, and liver fibrosis. The co-culture of stellate cells by using the cell culture chamber according to the disclosure can, for example, enable the study of the mechanisms of fibrogenesis—in particular, by tests of potentially participating mediators or signal molecules.

The porous membrane between the first and the second channels is advantageously selected with a thickness from a range of 10 to 20 μm, and in particular from a range of 10 to 13 μm. For example, the membrane has a thickness of 12 μm. A membrane thickness above 20-50 μm makes the substance exchange and soluble factor exchange between the vascular and the hepatic chamber more difficult. In addition, the migration of immune cells into the liver tissue is made more difficult by high membrane thicknesses, and negatively influences the results of active ingredient tests. The membrane can preferably be made of a rigid material, such as polyethylene terephthalate (PET). Alternatively, the membrane can also be made of a flexible material, such as a thermoplastic elastomer (TPE) or an elastic polyurethane (TPU). In these cases, the membrane can be designed with a thickness of 10 to 75 μm, and preferably 10 to 50 μm. The pore size of the membrane is advantageously in a range of 0.4 am to 8 μm. It has been found that pore sizes of more than 3 μm, for example, 5 am, 6 μm, 7 μm, or 8 μm, facilitate the exchange of soluble factors and the migration of immune cells into the hepatic channel. The membrane can have an additional surface structuring which can be produced, for example, by laser structuring. Such a structure can be advantageously adjusted to manipulate cell growth and/or to manipulate the flow-mechanical properties of the membrane (for example, to establish predetermined flow profiles).

Furthermore, the membrane and/or the channels can be plasma-treated with oxygen plasma, for example. Non-polar materials, perhaps including plastics with long polymer chains, have very low surface energy (generally 20-40 mN/m; by way of comparison, PDMS has only 20 mN/m), which does not match the high surface tension of aqueous liquids as they occur regularly in the cell culture. Polar materials such as PET (44 mN/m) and PBT (48 mN/m) are slightly higher. The surface energy of plastics can be made of a disperse and a polar component. Especially the polar component of the surface energy is very low. The plasma treatment significantly increases the surface energy (up to 80-90 mN/m), primarily of its polar component. In addition, the surfaces are freed of any existing release agents and functionalized. In this process, the treatment with oxygen plasma produces reactive oxygen species which break up polymer bonds and make them vulnerable to chemical biological interactions, or insert additional hydroxy groups into the PBT polymer. As a result, the membrane and/or channel surface becomes more hydrophilic, which results in improved coatability with biological components (such as collagen, fibronectin, laminin), improved cell adhesion, and a significant reduction in the adsorption of hydrophobic substances.

The plasma treatment can be preceded by a cleaning step in that the cell culture chamber is incubated in isopropanol or ethanol for 10 min, for example. The actual plasma treatment of the dried chambers can take place in particular in a low-pressure plasma method. For this purpose, the (cleaned) cell culture chambers are placed in a vacuum chamber of, for example, borosilicate glass and treated at a low pressure of <100 Pa (<1 mbar) for up to 20 min. with oxygen plasma. The oxygen plasma can be generated by short-wave excitation with a high-frequency generator at 13.56 MHz and up to 200 W power.

The first and the second channels are arranged one above the other in an advantageous operating state of the cell culture chamber. This means that the first channel is arranged above the second channel with respect to the effect of gravity. Such an arrangement facilitates, for example, the observation of the cells via an inverse microscope so that the required free space for manipulating the cell culture chamber is maintained above the cell culture chamber. Included in this understanding are also embodiments of the cell culture chamber in which the first channel is arranged at an angle up to 600 relative to the direction of action of gravity over the second channel, which can be achieved, for example, by an oblique arrangement of the membrane between the first and second channels, as well as operating positions of the cell culture chamber which are at an angle up to 600 relative to the direction of action of gravity.

The disclosure provides a model system in which, under specific, body-like culture conditions—in particular, human LSEC—alone and in co-culture with other liver-specific cell types, both their markers and their functional properties are preserved for at least 4 days, and in particular for up to 14 days. Accordingly, the expression of the following LSEC markers in the model system is detectable for up to 14 days: CD31, CD206, vWf, factor VIII, LSECtin, L-SIGN, LYVE-1, stabilin-2, VAP-1, FcRn, MHC I, and MHC II.

This progress is achieved, in addition to the material properties of the PBT, by further optional improvements. Accordingly, in an embodiment of the device according to the disclosure, at least one side surface of the membrane can be coated with a mixture containing fibronectin, and/or collagen I, and/or collagen IV. The term, “coated,” includes that the fibronectin, and/or collagen I, and/or collagen IV is/are chemically bound (covalently or non-covalently) to the membrane. In this case, the fibronectin is present in a concentration of 0.5 to 5 μg/mL, the collagen I in a concentration of 100 to 300 μg/mL, and the collagen IV in a concentration of 100 to 300 μg/mL. In additional embodiments, the coating can be present on both side surfaces of the membrane. For certain uses, it can be advantageous if no collagen I is contained, since this is increasingly formed in the liver, for example, during fibrosis, and therefore does not represent a typical physiological state.

In additional embodiments of the disclosure, it is possible for at least one of the side surfaces of the membrane to be coupled to dextran chains to which fibronectin peptides, or RGD peptides, or vitronectin peptides, or bone sialoprotein peptides are bound with an RGD sequence (arginine glycine aspartic acid). The RGD sequence serves in particular to mediate cell adhesion.

It is also possible for at least one of the side surfaces of the membrane to be coupled to heparin chains to which fibronectin peptides or FGF peptides are bound with an RGD sequence or RGD peptides alone, or vitronectin peptides with an RGD sequence, or bone sialoprotein peptides with an RGD sequence. FGF peptides are peptides of fibroblast growth factor.

If a previously mentioned coating of the membrane is present, the LSEC can have the following markers up to 14 days in the model system: CD32b, CD31, vWf, factor VIII, LSECtin, L-SIGN, stabilin-2, VAP-1, LYVE-1, FcRn, MHC I, and MHC II.

The cell culture chamber according to the disclosure can be used in a method for cultivating human or animal cells such as cells of the mouse, the rat, the pig, or the dog, et cetera.

In this method, a culture medium is conveyed permanently or temporarily through at least one of the channels.

In the method according to the disclosure, the cells are preferably cultured on an apical side of the membrane. In the sense meant herein, “apical” means that the cell culture is flushed by the culture medium on the side facing the membrane. In particular, this is the side surface of the membrane directed upwards, that is, against the force of gravity. Apically cultured cells accordingly grow on the membrane side which is flushed by the culture medium.

However, the culture medium can also be brought into contact basolaterally with the cell culture. A basolateral contact with apically cultured cells accordingly exists when there is contact via the porous membrane.

Particularly preferably, the cell culture chamber according to the disclosure can be used in a method for cultivating LSEC. The LSEC can be cultured alone or in co-culture with other liver-specific cell types; a co-culture with other liver-specific cell types is referred to herein as a liver model.

For the culture of LSEC, a vascular culture medium (LSEC medium; ECM) is used that is preferably apically applied in the first channel. This can include a basal endothelial cell basic medium, such as M199 or MCDB, which contains 0-20% human serum and/or 0-20% FCS (fetal calf serum), 0-10 ng/mL HGF (hepatocyte growth factor), 0-10 ng/mL EGF (epidermal growth factor), 0-10 ng/mL bFGF (basic fibroblasts growth factor), 0-20 ng/mL IGF-I, 0-5 ng/mL VEGF (vascular endothelial growth factor), 0-50 μM hydrocortisone, 0-5 mM ascorbic acid, and/or 0-4% ITS (insulin/transferrin/selenium) and heparin (0-5 U/mL). In particular for co-culture with hepatocytes and/or stellate cells, a preferably hepatic basic medium that is applied basolaterally in the second channel can be used, such as Williams E medium, which contains 0-20% FCS (fetal calcium serum) and/or 0-20% human serum, 0-5 mM glutamine or Glutamax, 0-10 μg/mL insulin, 0-4% MCB, 0-5 μM dexamethasone, 0-50 μM hydrocortisone, and 0-5% DMSO. The hepatic culture medium can be inoculated with hepatocytes and/or stellate cells. The culture medium can advantageously be produced free of additives of animal origin. Of course, it is equivalent and also possible to proceed conversely and apply the vascular culture medium to the second channel for the culture of LSEC and the hepatic basic medium in the first channel.

It has been found that the functionality of an LSEC cell culture can be prolonged if it is cultured in co-culture with other cells (again, both apical and basolateral). In particular, co-cultured cells such as hepatocytes, stellate cells, and immune cells such as, for example, Kupffer cells/macrophages have an advantageous effect on the preservation of the functionalities compared to cell cultures without a co-culture. It is advantageous here if the cells used for constructing the liver model have the identical HLA (human leukocyte antigen) status.

Advantageously, the shear rate or flow rate of the culture medium in at least one of the channels is selected such that it is comparable to the shear rates occurring in the body. For example, vascular conditions (of a blood vessel) are simulated in the first channel. Hepatic conditions can be simulated in the second channel. This is achieved, for example, in that an apical culture medium with a shear rate applied to the liver sinusoidal selected from a range of >0.2 to 1 dyn/cm², and preferably between 0.5 and 0.8 dyn/cm², is conveyed through the first channel, and a basolateral culture medium with a shear rate selected from a range of greater than zero to 0.2 dyn/cm² is conveyed through the second channel. Particularly in the case of a cell culture with LSEC, the shear rate in the first channel is particularly preferably adjusted to about 0.7 dyn/cm², which corresponds to the physiological vascular conditions in the liver. The shear rate adjusted in the second channel largely avoids unintentional settling of, for example, transmigrated immune cells in the direction of gravity away from the membrane and accumulation of metabolic end products of hepatocytes. The dimensions of the channels can accordingly be adapted, and therefore different. For example, the second channel can have a smaller width and/or a greater height than the first channel. Of course, it is equivalent and also possible to proceed conversely and to simulate hepatic conditions in the first channel and vascular conditions in the second channel.

The oxygen content in the culture medium—in particular, in the apical medium—is advantageously adjusted to a value of 15% at the channel inlet and 3 to 5% at the channel outlet. The use of the cell culture chamber according to the disclosure and the culture medium used, the stated shear rates, a connection of two channels of a cell culture chamber or several cell culture chambers to one another, and optionally the adjustment of hypoxia conditions, for example, by arranging the cell culture chamber(s) in a hypoxia cabinet, allow the adjustment of the mentioned oxygen content.

The cell culture chamber according to the disclosure can be used in additional applications, for example, for cultivating intestinal cells, lung cells, and/or lung alveoli cells, and the in vitro production of cell layers or organ models of these cell types.

To establish a liver model, the membrane can be coated in an upstream step with proteins of the extracellular matrix and apically colonized with LSEC—preferably in co-culture with Kupffer cells. The membrane can alternatively or additionally be colonized with hepatocytes in particular basolaterally, and advantageously in co-culture with stellate cells. Furthermore, immune cells such as monocytes, macrophages, T-cells, B-cells, and neutrophils can be added to the apical culture medium or culture media and flushed into the cell culture chamber. This makes it possible to perfuse defined immune cell populations via the apical side and to check the influence of these immune cells on the liver model. The term, “flushed,” includes that flushed cells can circulate through the vascular chamber and adhere to surfaces, that is, can settle on the endothelial cell layer or the membrane, and in particular at or near LSEC cells.

To establish an intestinal model, the membrane can be coated in an upstream step with proteins of the extracellular matrix, and preferably apically colonized with intestinal endothelial cells—preferably in co-culture with immune cells. The membrane can alternatively or additionally be colonized in particular basolaterally with intestine epithelial cells, and/or with smooth muscle cells, and/or immune cells. Furthermore, immune cells such as monocytes, macrophages, T-cells, B-cells, and neutrophils can be added to the culture medium or culture media. The intestinal models can be cultivated apically or basolaterally with shear rates of 0-3 dyn/cm². In addition, microorganisms (for example, parts of the microbiome) can be added to the basolateral culture medium in order, for example, to examine the influence of such microorganisms on the intestinal model.

To establish a lung model, the membrane can be coated in an upstream step with proteins of the extracellular matrix and preferably apically colonized with pulmonary epithelial cells (for example, small airway epithelial cells and alveolar epithelial cells)—preferably in co-culture with immune cells. The membrane can be colonized in particular basolaterally with pulmonary endothelial cells and also in an apically/basolaterally reversed arrangement. Furthermore, immune cells such as monocytes, macrophages, T-cells, B-cells, and neutrophils can be added to the culture medium or culture media. The lung models can be cultivated apically or basolaterally with shear rates of 0-2 dyn/cm². In addition, the epithelial cells can be cultured without medium after reaching a high confluence in order to form an air-liquid interphase, as in the body.

In order to examine physiological effects of active substances, or signal molecules, or microorganisms, et cetera, on the cultured cells, cell layers, or organ models via a cell culture chamber according to the disclosure, active substances or molecules or microorganisms can be added to the cultured cells, cell layers, or organ models by flushing them into the cell culture chamber. For example, when a desired colonization status is reached, an active ingredient to be investigated or several active ingredients to be investigated can be added to the culture medium or another liquid carrier medium in desired dosages apically or basolaterally, and the cell culture can be flushed therewith (perfused). For example, properties of the culture medium emerging from the particular channel can be recorded as measured values. With a suitable design of the cell culture chamber and its optical properties, microscopic investigations, for example, direct and/or fluorescence-based observations of the adherent cells growing on the membrane and/or the co-cultured cells, can be carried out. It is likewise possible to proceed with signal molecules or microorganisms.

A great advantage of the disclosure is the possibility of reconfiguring different conditions in the channels. In particular, the conditions of a blood vessel can be simulated in one of the two channels, which can be referred to as vascular conditions. With respect to the membrane, this channel can be referred to as a vascular side. In the other channel, for example, the conditions in a liver tissue can be modeled, which can be referred to as hepatic conditions or as the hepatic side with respect to the membrane. On the hepatic side, which is provided in particular by the lower channel, a low, but sufficiently high shear rate is generated by the culture medium, which considerably reduces the unwanted settling of cells away from the membrane. Due to their material properties, the channels of the cell culture chamber according to the disclosure also have a low adsorption of compounds which are contained in a culture medium. Advantageously, this has little effect on the results of experiments.

### BRIEF DESCRIPTION OF DRAWINGS

The invention will now be described with reference to the drawings and graphics of performed experiments wherein:

FIG. 1 shows a schematic representation of a cell culture chamber according to the disclosure with two channels and a membrane colonized in co-culture;

FIG. 2 shows a schematic representation of a second embodiment of a cell culture chamber according to the disclosure with four channels in an exploded view;

FIG. 3A shows a graphical representation of the results of an experiment on the adsorption effect of PBT in two cell culture chambers according to the disclosure;

FIG. 3B shows a graphical representation of the results of another experiment on the adsorption effect of PBT in two cell culture chambers according to the disclosure;

FIG. 4 shows a graphical representation of the results of an experiment on the release of LDH (lactate dehydrogenase) over a period of 10 or 14 days compared with a cell culture chamber according to the disclosure and the conditions of the cell lysis;

FIG. 5 shows a graphical representation of the results of an experiment on the release of ASAT (aspartate aminotransferase) over a period of 10 or 14 days compared with a cell culture chamber according to the disclosure and the conditions of the cell lysis;

FIG. 6 shows a graphical representation of the results of an experiment on the detection of the synthesis and release of albumin over a period of 10 or 14 days compared to a cell culture chamber according to the disclosure and the conditions of the cell lysis;

FIG. 7 shows a graphical representation of the results of an experiment on the detection of the synthesis and release of urea over a period of 10 or 14 days compared to a cell culture chamber according to the disclosure and the conditions of the cell lysis;

FIG. 8 shows a graphical representation of the results of an experiment on the detection of interleukin-1 beta over a period of 10 or 14 days compared to culturing in a cell culture chamber according to the disclosure and with the addition of LPS;

FIG. 9 shows a graphical representation of the results of an experiment on the detection of interleukin-10 over a period of 10 or 14 days compared to culturing in a cell culture chamber according to the disclosure and with addition of LPS; and,

FIG. 10 shows a graphical representation of the results of an experiment on the detection of interleukin-6 over a period of 10 or 14 days compared to culturing in a cell culture chamber according to the disclosure and with addition of LPS.

### DETAILED DESCRIPTION

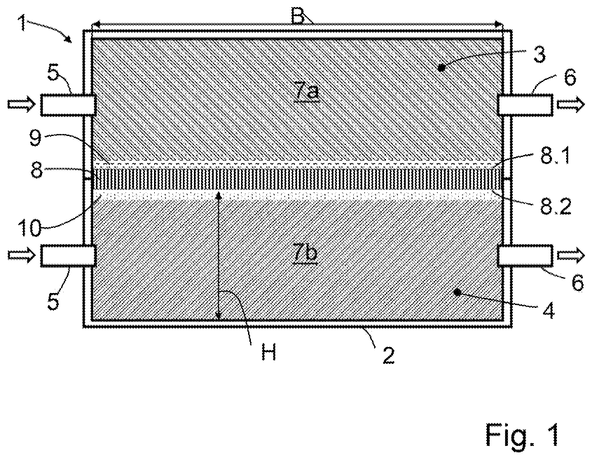

A cell culture chamber 1 according to the disclosure according to FIG. 1 includes a housing 2 which is made of polybutylene terephthalate (PBT) at least in the regions of a first channel 3 and a second channel 4. Each of the channels 3, 4 has an inlet 5 and an outlet 6 which serve for the inflow and discharge, respectively, of a culture medium 7 into and out of the particular channels 3, 4. The first channel 3 and the second channel 4 are separated from one another by a porous membrane 8. In the first channel 3, which at the same time represents an apical region of the cell culture chamber 1, a cell culture 9, for example, of liver sinusoidal endothelial cells, is located on a side surface, facing the first channel 3, of the membrane 8, which side surface is referred to as apical side 8.1 of the membrane.

An optionally present co-culture 10 containing hepatocytes is located on a side surface, facing the second channel 4, of the membrane 8, which side surface is referred to as the basolateral side 8.2 of the membrane. The membrane 8 is, for example, 12 μm thick and made of PET.

The channels 3, 4 each have a width B, a height H transverse thereto, and a depth (not designated) perpendicular to the plane of the drawing.

In another embodiment of the disclosure, vascular conditions of, for example, a blood vessel are simulated in the first channel 3 arranged at the top relative to the gravitational effect, while in the second channel 4, hepatic conditions are simulated.

The shear rate in the model is adjusted for the vascular side and the hepatic side in that an apical culture medium 7a with a shear rate of 0.7 dyn/cm² is conveyed through the first channel 3, and a basolateral culture medium 7b with a shear rate of >0 to 0.2 dyn/cm² is conveyed through the second channel 4.

In the embodiment, the coating of the membrane 8 consists of a mixture of fibronectin/collagen I and collagen IV (fibronectin 5 μg/mL, collagen I 0.3 mg/mL, collagen IV 100 μg/mL). The cell cultures 9, 10 are cultivated in cell-type-specific and species-specific media with specific formulations. The apical culture medium 7a of the first channel 4 (ECM) contains 10% human serum and growth factors, antioxidants, and ITS in suitable concentrations familiar to a person skilled in the art. The basolateral medium 7b of the second channel 4 contains growth factors, a collagen IV/I mixture, and ITS in suitable concentrations familiar to a person skilled in the art.

The addition of specific media from the basolateral side can be replaced by the culture of hepatocytes and, optionally, stellate cells in cell-specific medium 7b in the second channel 4. In addition, macrophages have a positive influence on the properties/functionality of the cell culture 9 on the apical side (first channel 3).

A pump unit for each channel 3, 4 for controlled conveying of the particular culture medium 7, 7a, 7b and a controller for controlling the pump units are not shown in more detail. In addition, various sensors (for example, for oxygen, pH, lactate, TEER) can be present, via whose measured values the cell cultures 9 and 10 can be investigated. The sensors can be connected to the control unit in order, for example, to regulate the composition and/or the shear rate or flow rate of the particular culture medium 7, 7a, 7b as a function of the detected measured values.

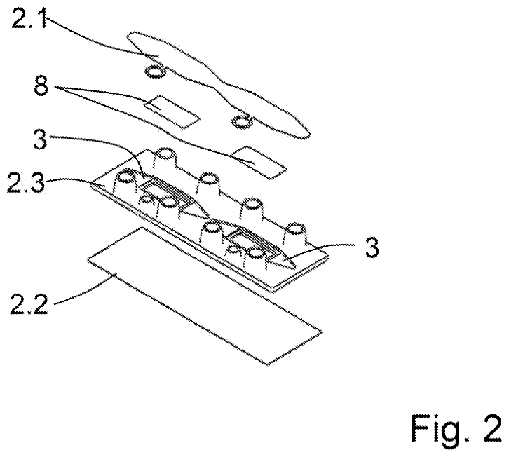

A second embodiment of a cell culture chamber 1 according to the disclosure with two first channels 3 and two second channels 4 (not visible) is shown in FIG. 2 in an exploded view. The housing 2 is formed from an upper cover 2.1, a lower cover 2.2, and a central piece 2.3 in the form of an injection-molded piece. The center piece 2.3 contains the formations of the channels 3 and 4, which are each separated from one another by an inserted membrane 8.

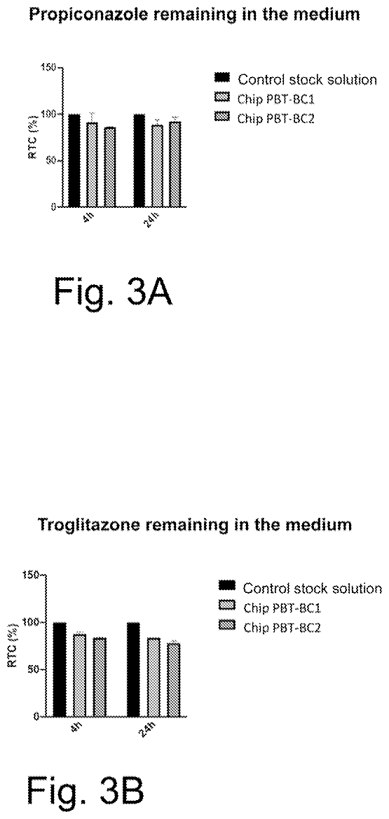

FIGS. 3A and 3B show results of experiments on the adsorption effect of PBT in two cell culture chambers 1 according to the disclosure of different designs. The designation, “Chip PBT-BC1,” denotes a first embodiment of the cell culture chamber 1, and “Chip PBT-BC2” a second embodiment. The first embodiment has a width B of 34.6 mm, a depth T of 6.56 mm, and a height H of 0.7 mm for the first channel 3. The second channel 4 has the dimensions of 44.8 mm, 3.6 mm, and 0.8 mm (W/D/H). The corresponding dimensions for the second embodiment, “Chip PBT-BC2,” of the cell culture chamber 1 are 34.6 mm, 8.06 mm, and 0.7 mm (W/D/H) for the first channel 3 and 49.4 mm, 5.61 mm, and 1.0 mm (B/T/H) for the second channel 4.

The graph in FIG. 3A shows the percentage (RTC—ratio to control) of propiconazole remaining and detectable in the culture medium 7 (log P 3.72) after an incubation time of 4 hours and after 24 hours. In comparison, a control in the form of a stock solution (100 μM) with propiconazole is given. The graph in FIG. 3B shows the percentage fraction of the troglitazone remaining in the culture medium 7 (log P 3.60). Also in the example of FIG. 3B, samples were taken after 4 hours and after 24 hours incubation time, which were compared with a control in the form of a troglitazone stock solution (64 μM). In each case, it can be seen from the two graphics that, in both embodiments of the cell culture chamber 1 according to the disclosure, more than 85% of the propiconazole (FIG. 3A) and more than 80% of the troglitazone (FIG. 3B) in the culture medium 7 could be detected even after 24 hours.

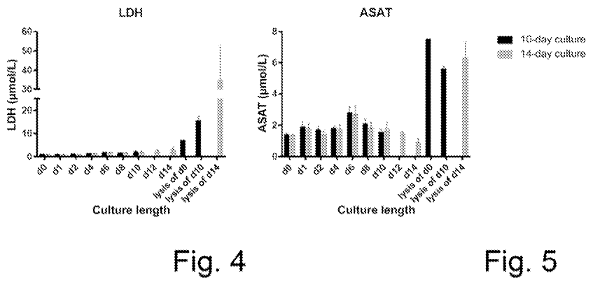

FIGS. 4 and 5 show the time profiles of the release of LDH (lactate dehydrogenases; FIG. 4) or of ASAT (aspartate aminotransferase; FIG. 5) of liver sinusoidal endothelial cells cultured in a cell culture chamber 1 according to the disclosure over a period of 10 or 14 days in co-culture with macrophages and hepatocytes compared to cell lysis. LDH and ASAT are enzymes that are released when there is cell damage and are therefore suitable for assessing the vital condition of an organ or tissues and cells. A significantly lower release of LDH and ASAT by the cultured cells compared to cell lysis can be seen even after 14 days.

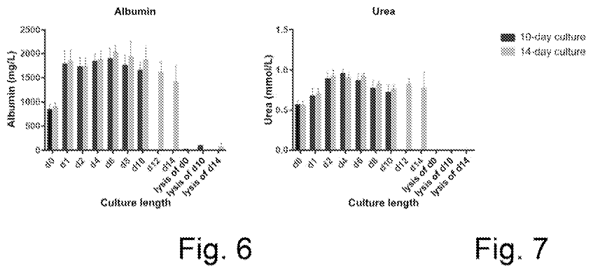

FIGS. 6 and 7 show the concentrations of albumin (FIG. 6) and urea (FIG. 7) of liver sinusoidal endothelial cells cultured in a cell culture chamber 1 according to the disclosure over a period of 10 or 14 days in co-culture with macrophages and hepatocytes compared to cell lysis. Albumin and urea can be regarded as an expression of existing and intact synthesis processes in liver tissue or hepatocytes. It can be seen that, both in the 10-day cultures and in the 14-day cultures, concentrations above 1,300 μg/L albumin or more than 0.7 mmol/L urea can also be detected after 10 or 14 days.

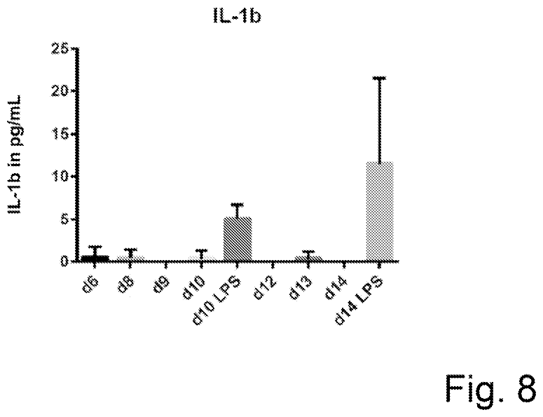

FIG. 8 shows the results of an experiment on the detection of interleukin-1 beta (IL-1b) over a period of 10 or 14 days. In addition, lipopolysaccharide (LPS) was added to some samples at the tenth day and the fourteenth day to check the activatability of immune cells (macrophages) contained in the liver model, and the respective reactions were shown. The addition of LPS as a so-called pyrogenic substance represents a test for endotoxins that is customary in the art. Immune cells such as macrophages respond to this stimulus with increased release of cytokines. The results show a distinct increase in the concentration of IL-1beta after the addition of LPS. It can be deduced from this that macrophages in co-culture with the investigated liver sinusoidal endothelial cells and hepatocytes were capable of reacting with an immune response to an endotoxin even after 10 or after 14 days. This is important for being able to detect immune cell-mediated side effects of drugs.

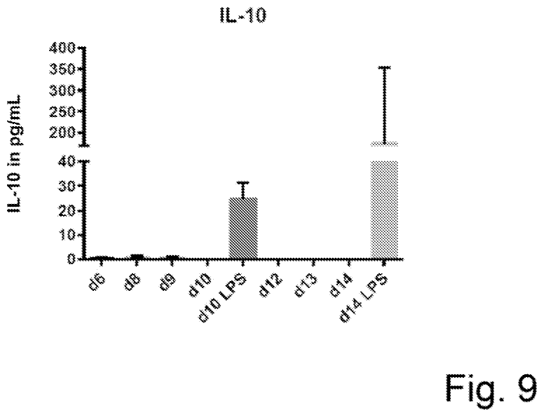

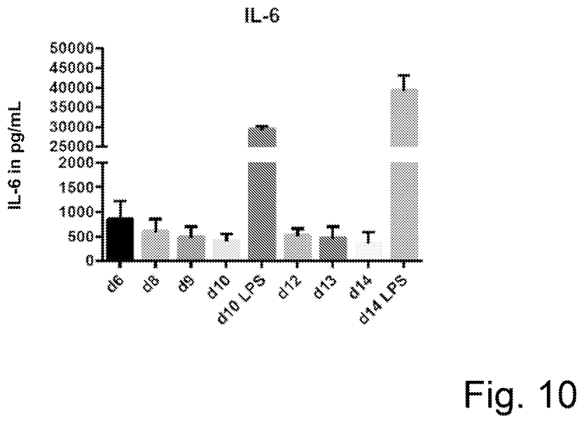

In principle, the same results are obtained for interleukin-10 (IL-10; FIG. 9) and interleukin-6 (IL-6; FIG. 10).

It is understood that the foregoing description is that of the preferred embodiments of the invention and that various changes and modifications may be made thereto without departing from the spirit and scope of the invention as defined in the appended claims.
1 Cell culture chamber
2 Housing
2.1 Upper cover
2.2 Lower cover
2.3 Center piece
3 First channel
4 Second channel
5 Channel inlet
6 Channel outlet
7 Culture medium
7a Apical culture medium
7b Basolateral culture medium
8 Membrane
8.1 Apical side of the membrane
8.2 Basolateral side of the membrane
9 Cell culture
10 Co-culture
B Width
H Height

1. A cell culture chamber, comprising:
a first channel comprising an inner wall, through which a first flow can pass;
a second channel comprising an inner wall, through which a second flow can pass; and
a porous membrane;
wherein the first and second channels are arranged one above the other and are separated from one another by the membrane,
wherein the membrane has a first side surface oriented towards the first channel and a second side surface oriented towards the second channel,
wherein the first and second side surfaces form a cell substrate, and
wherein the inner walls of the first channel and the second channel consist of polybutylene terephthalate (PBT).

2. The cell culture chamber according to claim 1, wherein the membrane has a thickness of 10 μm to 75 μm.

3. The cell culture chamber according to claim 1, wherein the membrane has a thickness of 10 μm to 50 μm.

4. The cell culture chamber according to claim 1, wherein the membrane has a thickness of 10 μm to 13 μm.

5. The cell culture chamber according to claim 1, wherein the membrane consists of polyethylene terephthalate (PET), or a thermoplastic elastomer (TPE), or an elastic polyurethane (TPU).

6. The cell culture chamber according to claim 1, wherein the at least one side surface of the membrane has an additional surface structure.

7. The cell culture chamber according to claim 1, wherein at least one of the first and second side surfaces of the membrane is plasma-treated, and/or wherein at least one of the first or the second channels is plasma-treated.

8. The cell culture chamber according to claim 1, wherein at least one of the first and second side surface of the membrane is coated with a mixture comprising at least one of: 0.5 μg/mL to 5 μg/mL fibronectin, 100 μg/mL to 300 μg/mL collagen I, and 100 μg/mL to 300 μg/mL collagen IV.

9. The cell culture chamber according to claim 1, wherein at least one of the first and second side surfaces of the membrane is coupled to dextran chains to which fibronectin peptides, arginine-glycine-aspartic acid (RGD) tripeptides, vitronectin peptides, or bone sialoprotein peptides are bound with an RGD sequence.

10. The cell culture chamber according to claim 1, wherein at least one of the first and second side surfaces of the membrane is coupled to heparin chains to which fibronectin peptides or fibroblast growth factor (FGF) peptides are bound with an arginine-glycine-aspartic acid (RGD) sequence, RGD peptides alone, vitronectin peptides with an RGD sequence, or bone sialoprotein peptides with an RGD sequence.

11. The cell culture chamber according to claim 1, wherein human collagen I and/or human collagen IV with a concentration of more than 100 μg/mL is coupled to at least one of the first or second side surfaces of the membrane.

12. A method for culturing human or animal cells, which comprises:
providing the cell culture chamber according to claim 1,
providing the human or animal cells,
adding the human or animal cells to the culture chamber through one of the channels of the culture chamber, and
incubating the cells in the culture chamber under culturing conditions.

13. The method according to claim 12, further comprising conveying a culture medium through the first channel and/or the second channel into the culture chamber, wherein the cells are cultured on an apical side of the membrane.

14. The method according to claim 12, further comprising conveying a culture medium through the first channel and/or the second channel into the culture chamber, wherein the cells are cultured on a basolateral side of the membrane.

15. The method according to claim 12, wherein the cultured cells are liver sinusoidal endothelial cells.

16. The method according to claim 12, wherein vascular conditions are simulated in the first channel and hepatic conditions are simulated in the second channel, by:
conveying through the first channel an apical culture medium possessing a shear rate of more than 0.2 dyn/cm² to 1 dyn/cm², and
conveying through the second channel a basolateral culture medium possessing a shear rate of greater than zero up to and including 0.2 dyn/cm².

17. The method according to claim 16, wherein the apical culture medium possesses a shear rate of 0.5 dyn/cm² to 0.8 dyn/cm².

18. The method according to claim 12, wherein vascular conditions are simulated in the second channel and hepatic conditions are simulated in the first channel, by:
conveying through the first channel a basolateral culture medium possessing a shear rate of greater than zero up to and including 0.2 dyn/cm², and
conveying through the second channel an apical culture medium possessing a shear rate of more than 0.2 dyn/cm² to 1 dyn/cm².

19. The method according to claim 18, wherein the apical culture medium possesses a shear rate of 0.5 dyn/cm² to 0.8 dyn/cm².

20. The method according to claim 12, in which a culture medium is conveyed through at least one of the first and second channels, wherein the oxygen content in the culture medium is adjusted at an inlet of the channel to a value of 15%, and wherein the oxygen content in the culture medium is adjusted at an outlet of the channel to a value of between 3% and 5%.

21. The method according to claim 15, in which the liver sinusoidal endothelial cells are co-cultured with one or more of Kupffer cells, hepatocytes, and stellate cells.

22. The method according to claim 12, wherein the cultured cells are one or more of intestinal endothelial cells, intestinal epithelial cells, and intestinal smooth muscle cells.

23. The method according to claim 12, wherein the cultured cells are pulmonary epithelial cells and/or pulmonary endothelial cells.

24. The method according to claim 12, further comprising flushing an active substance and/or a signal molecule into the cell culture chamber.

25. The method according to claim 12, further comprising flushing immune cells and/or microorganisms into the cell culture chamber.
